(19) United States
(12) Patent Application Publication
(10) Pub. No.: US 2023/0083702 A1
(43) Pub. Date: Mar. 16, 2023

(54) PLASTIC LASER WELDING FOR STEERABLE CATHETER TIP

(71) Applicants: CANON U.S.A., INC., MELVILLE, NY (US); CANON VIRGINIA, INC., Newport News, VA (US)

(72) Inventors: Nathaniel Robert Lau, Boston, MA (US); Showna Chang, Arlington, MA (US); Takahisa Kato, Brookline, MA (US); Joel C. Johnson, Franklin, VA (US); Terry Speed, Toana, VA (US)

(21) Appl. No.: 17/799,472

(22) PCT Filed: Feb. 22, 2021
(86) PCT No.: PCT/US2021/018953

§ 371 Date: Aug. 12, 2022

Related U.S. Application Data
(60) Provisional application No. 62/979,930, filed on Feb. 21, 2020.
(60) Provisional application No. 63/132,743, filed on Dec. 31, 2020.

(51) Int. Cl. B29C 65/16; B29C 65/00

(52) U.S. Cl. CPC B29C 65/1635; B29C 66/739

(57) ABSTRACT

The present disclosure relates to methods of manufacture, apparatus, and fixtures. An apparatus comprising an inner liner having a hollow chamber extending the length of the inner liner, at least two guide rings disposed collectively along the inner liner, and at least one lumen portion extending through each of the at least two guide rings and being parallel with the hollow chamber, wherein the at least two components are fixed by welding is provided. Further provided is a fixture and a method of manufacture.

### PRIORITY

The present application claims the benefit of priority to U.S. Provisional Patent Application Nos. 62/979,930, filed on Feb. 21, 2020, and 63/132,743, filed on Dec. 31, 2020, which are incorporated herein by reference in their entireties.

### FIELD OF THE DISCLOSURE

The present disclosure relates to methods of manufacture, apparatus, and fixtures. More particularly, the subject disclosure is directed to methods of manufacture and to a bendable medical device having a hollow chamber and ring guides along the device.

### BACKGROUND INFORMATION

Bendable medical devices such as endoscopic surgical instruments and catheters are well known and continue to gain acceptance in the medical field. The bendable medical instrument generally includes a flexible body commonly referred to as a sleeve or sheath. One or more tool channels extend along (typically inside) the flexible body to allow access to a target located at a distal end of the flexible body.

Some bendable devices have an inner liner, an outer liner, and optionally other components within the device that provide for actuation of the flexible body. However, for bendable devices where a small diameter is required and where a large degree of curvature is required at the distal end of the device, such configurations are not particularly useful since the various liners and components add rigidity and prevent the device from bending sufficiently. Thus, the use of guide rings which are arranged along the device to guide the wires used to control the device. See, for example, U.S. Pat. No. 9,144,370. Another example of a bendable medical device with the guide rings is described in WO 2018/204202. In this device, the guide rings are attached to the inner liner, or backbone, by adhesive. However, the use of adhesive in fabrication is not always preferred since adhesive can be difficult to apply, can add unwanted material, and the spacing of the guide ring structure cannot be controlled. In particular, the spacing of the guide rings directly effects the functionality of the bendable medical device. When adhesives are first applied, they can often act like a lubricant, causing the components to be more prone to movement until the adhesive bond beings to form, making it more likely that the guide ring spacing may be altered or inconsistent. It can also be important that there is no adhesive between the rings or blocking small lumens within the rings in order for the catheter to function properly. If adhesive is placed between the rings or if it blocks the lumens, the bendable medical device can lose some of the bendable degrees of freedom. Adhesives are typically applied manually, and due to the sizes of catheter components, such manual processes can be difficult to complete without the mis-application of adhesive described previously. Additionally, the bond strength with adhesive can be inconsistent and lower than would be required for required catheter durability, as the loosening or displacement of any of the guide rings can also negatively impact the catheter function. Thus, there are needs for additional bendable devices and methods of manufacture to overcome these problems. There is a need to attach guide rings that can have short lengths and contain lumens within the guide rings with a set spacing along the device.

There are currently multiple additive and non-additive methods to attach components together. Non-additive methods such as ultrasonic and heat can be used, but the process can deform surrounding material and are not very localized, making it difficult to use on small diameter devices such as catheters. There are exceptions to this; EP 1234595 provides a balloon catheter having a plastic laser welding between a balloon and catheter body using an infrared wavelength of no more than 1580 nanometers (i.e., a ND:YAG laser or a low power diode laser). However, this works for a balloon, which is a thin, sheet-like material extending a length along the catheter that simply needs to be adhered. Thus, there is need to overcome the deficiencies as described herein to form bendable devices and methods of manufacture for devices having guide rings that need to be attached without changing the configuration of lumen sections running there through or for guide rings adhered at a known, spaced relationship.

### SUMMARY OF EXEMPLARY EMBODIMENTS

According to at least one embodiment of the invention, there is provided an apparatus comprising an inner liner having a hollow chamber extending the length of the inner liner; at least two guide rings disposed collectively along the inner liner; at least one lumen portion extending through each of the at least two guide rings and being parallel with the hollow chamber; wherein at least two components are fixed by welding. The apparatus may also comprise an outer liner which may be the distal portion of a catheter. The apparatus may additionally comprise an extrusion. In further embodiments, the at least two guide rings are welded to the inner liner, and/or the outer liner is welded to the at least two guide rings, and/or the extrusion is welded to the inner liner. In some embodiments, the apparatus also comprises a plurality of wires extending through the lumens, either through some or all of the guide rings or, if the apparatus has different sections, through at least one section of the guide rings.

According to other embodiments of the invention, there is provided a method of manufacture. The method comprises: combining a plurality of guide rings around the outside of an inner liner to create an assembly; placing the assembly on a fixture adapted to set a distance between each of the plurality of guide rings; and welding each of the plurality of guide rings to the inner liner. The plurality of guide rings is substantially transparent and contains at least one lumen portion. According to a further embodiment, the welding occurs through both lumen containing areas and non-lumen containing areas. According to still other embodiments of the invention, there is provided a laser welding system, which system may comprise a vision system, a laser generator, a transfer fiber, a beam shaper, a galvanometer head, a motorized fixture, and a controller in communication with the vision system, the motorized fixture and the laser generator, wherein the laser welding system is configured to weld two or more components of a steerable medical device. According to a further embodiment, the welding occurs through both lumen containing areas and non-lumen containing areas. In other exemplary embodiments, the system is configured to weld (i) an inner liner having a hollow chamber extending the length of the inner liner and at least two guide rings disposed collectively along the inner liner; (ii) an outer liner to one or more guide rings; and/or (iii) an extrusion to the inner liner.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1 depicts a side perspective three-dimensional view of an exemplary medical device, according to one or more embodiment of the subject apparatus, method or system.

FIG. 2 depicts a side perspective three-dimensional view of an exemplary medical device, according to one or more embodiment of the subject apparatus, method or system.

FIG. 3 is diagram of an embodiment showing a transparent rings welded to a light absorbing inner lumen in an exemplary medical device, according to one or more embodiment of the subject apparatus, method or system.

FIG. 4 is diagram of an embodiment showing a weld configuration in a method of forming the medical apparatus, according to one or more embodiment of the subject apparatus, method or system.

FIG. 5 is diagram of an embodiment showing another weld configuration in a method of forming the medical apparatus, according to one or more embodiment of the subject apparatus, method or system.

FIG. 6 depicts a side perspective three-dimensional view of an exemplary medical device, according to one or more embodiment of the subject apparatus, method or system.

FIG. 7 is an exemplary laser configuration that may be used with the present invention.

FIG. 8 is an exemplary computer configuration that may be used with the present invention.

FIG. 9 is a diagram depicting exemplary laser patterns that may be used with the present invention.

FIGS. 10A and 10B are side perspective, three-dimensional views depicting differences in weld length, according to one or more embodiment of the subject apparatus, method or system.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

The apparatus, methods of manufacture of the apparatus, systems, and manufacture configurations as described herein relates to catheter manufacturing and the attachment of components, such as (i) guide rings, to the inner liner that forms the inner lumen of, for example, a catheter; (ii) guide rings to the outer liner that forms the outer portion of, for example, a catheter, and/or (iii) guide rings to an extrusion that forms a portion of, for example, a catheter, and to the medical apparatus formed by the manufacture.

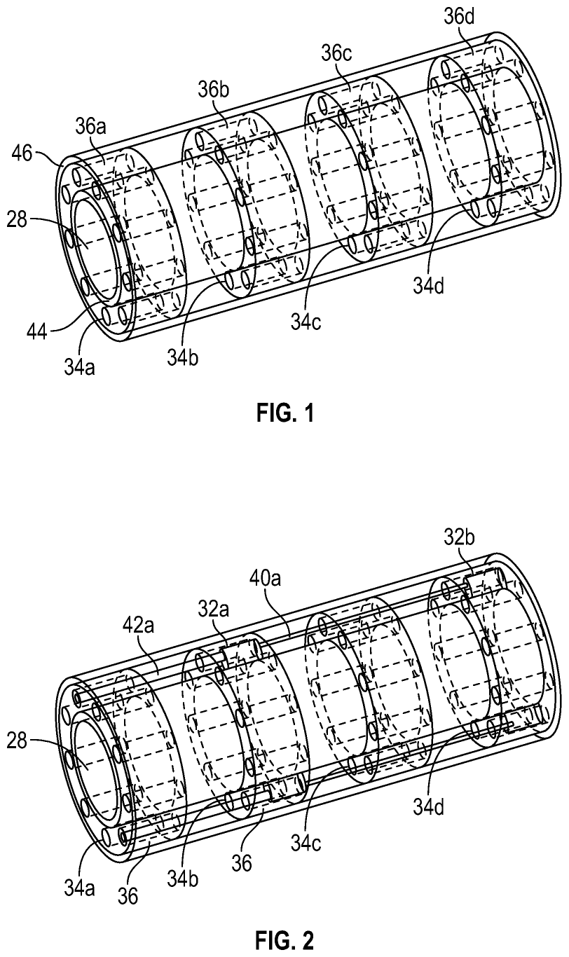

In some embodiments, the distal portion of the medical apparatus is shown in FIGS. 1 and 2, which depict side perspective three-dimensional views of an exemplary bendable medical device, wherein the bendable medical device includes at least two guide rings 36a, b (four are shown—36a, b, c, d) confined within the bendable body 26, wherein the guide rings 36 are configured a distance apart from one another and do not contact one another.

The bendable body 26 comprises an inner liner 44 and an outer liner 46, which provides bendable support to the bendable body 26 while retaining the guide rings 36 in a constant position along the axial direction of the bendable body 26. Inside the inner liner 44 is a hollow chamber 28 extending the length of the inner liner. This hollow chamber 28 can be used, for example, as a tool channel or working channel of a catheter. Each guide ring 36 contains at least two lumens portions 34, and are configured to house the anchor segments 32a, b embedded into the guide rings 36. The space between adjacent guide rings, in cooperation with the resilient inner liner 44 and outer liner 46, allows the bendable body 26 to achieve a greater range of bending motion due to the open space between the guide rings 36.

The medical apparatus is configured and/or adapted for in vivo use, including with respect to size and maneuverability. The configuration of discrete sections and continuous outer liner 46 can be tuned to the required flexibility necessary for navigation to accommodate the anatomy of the patient. The spacing of the guide rings 36 may be increased or decreased depending on, for example, the range of bending, diameter, and structure of the bendable body 26. As shown, all guide rings 36 are equally spaced. However, in some embodiments, the spacing may vary. For example, there may be a different distance between the guide rings 36 between the different bendable segments of bendable body 26; the guide ring spacing within one bendable segment may be different from the guide ring spacing within another bendable segment; or the guide ring spacing may gradually increase or decrease along the bendable body 26. The diameter of the inner liner 44 and/or outer liner 46 may also be modified based on the patient anatomy. In some embodiments, the outer diameter of the bendable device (which is the outer diameter of the outer liner 46) is minimized to allow for both less invasive procedures and to allow for use of the medical device into smaller anatomy. For example, a device with an outer diameter of less than 5, 4, 3, or 2 mm can be used to maneuver within the segmental bronchus or sub-segmental bronchi of the lung (see, for example, U.S. Pat. Pub. 2019/0105468, herein incorporated by reference).

FIG. 2 further depicts an exemplary use of nested control wires, shown with wire 40a and 42a inside one set of lumen portions 34 in conjunction with the at least two guide rings 36. Furthermore, this embodiment allows multiple control wires 40a and 42a to be anchored at an anchor segment (32a and 32b) and to slidably move through the lumen portions 34 of the bendable body 26. While nested control wires are depicted as one exemplary configuration, alternative embodiments may also be used, including, for example, the use of separate wires, which may be anchored or fully slidable in one or more lumen portions, and wherein if anchored, the separate wires may be anchored in the same or different locations. Other suitable configurations of control wires will be apparent to those of skill in the art.

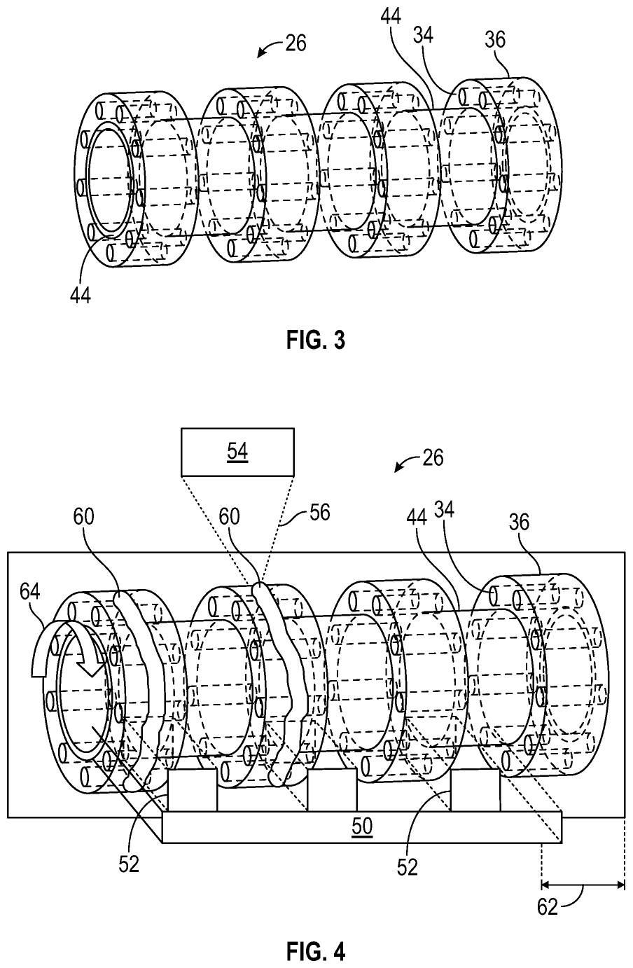

The plurality of guide rings 36 bonded to inner liner 44 will use laser welding instead of adhesives to create the bendable body 26 shown in FIG. 3. This distal portion of a medical device is shown without the optional outer liner or sheath 46. Multiple guide rings 36 are spaced along the inner liner 44 with all lumen portions 34 aligned such that a wire could be inserted through a lumen portion 34 of each of the ring guides 36. Thus, the bendable body 26 could also include wires 40a and 42a (not depicted) extending parallel to the hollow chamber 28, where each of the wires 40a and 42a extend through the lumen portions 34 of the guide rings 36. The array of lumen portions 34 extending proximally within the bendable body 26 and between the inner liner 44 and outer liner 46 creates an effective lumen that controls the placement of the wires. In some embodiments, nine or more wires are located within at least some of the guide rings 36.

In one exemplary embodiment, the outer liner 46 can be fixed to the guide rings 36 by laser welding. Such guide rings 36 can also be optionally fixed to the inner liner 44 by laser welding. In such an exemplary embodiment, the outer liner 46 may made of a material that is more transparent than that of the guide rings 36, and the guide rings 36 may be made of a material that is more transparent than that of the inner liner 44. Thus, the components to be welded may be made of materials that are of varying degrees of transparency, such that the outermost component to be welded is of a greater degree of transparency than the component it is to be fixed to. Depending on the number of components to be welded, there may be a total of 2, 3, 4, 5 or more different degrees (or levels) of transparency among all of the components to be welded. In one exemplary embodiment, any of the components of the bendable body that require fixation may have the fixation accomplished by welding.

To form the bendable body as shown in FIG. 3, a fixture 50 is used to hold the components in place during the welding process, as depicted in FIG. 4. One or more wires may be inserted into a lumen portion 34 of each guide ring 36 to aid in the rotational placement of the guide rings 36. Alternatively, the guide rings 36 may contain, for example, a cavity or indentation at the outer portion of the guide ring to align the plurality of guide rings along the bendable body. Fixture 50 may also contain one or more fixture spacers 52 to ensure the desired alignment of the guide rings 36 with respect to one another. Fixture spacers 52 may have the same or different dimensions, based on whether it is desired that the guide rings 36 have consistent spacing or another desired configuration. Laser welding may be used to first tack components in place to prevent the components from moving out of position within the fixture 50.

The guide rings are made of a less light absorbing material than the inner liner. Thus, when light is focused into the bendable body, the light will transmit through the guide ring and to the inner liner and allow for welding to take place at the interface between the two materials, for example, by through transmission welding. For example, the guide rings may be formed from extruded polyolefins, polyamides, polyesters, ethylene-vinyl acetate (EVA), or thermoplastic elastomers (TPEs such as Pebax®). The limitation of these materials is that they must be weldable. The inner liner may be formed from the same type of materials with the same or different hardness, but will have a great light absorption. In some examples, the inner liner contains a percentage of a dye or carbon black to increase the absorptivity of the inner liner, for instance, Pebax with 0.1 to 5%, for instance 0.1%, 0.5%, 1.0%, 2%, 3%, 4%, 5% Carbon Black. In certain embodiments, the inner liner may comprise at least 0.5% Carbon Black. In some embodiments a radiopaque additive is added (e.g., barium sulfate, bismuth subcarbonate, bismuth trioxide, bismuth oxychloride or tungsten) to provide both visibility by X-ray and preferred welding characteristics.

It is important that the guide rings absorb less light than the inner liner, and in some embodiments, the guide rings are substantially transparent at the wavelength of light used for the welding process. However, transparency is not required. It is important that the ring guide 36 is sufficiently transparent and the laser pattern sufficiently focused that the lumen portions 34 extending through the ring guide 36 is not melted or substantially deformed by the laser weld process. In some embodiments, it is preferred that none of the lumen portions are noticeably altered by the welding process that adheres the guide rings to the inner liner. The lumen portions would be noticeably altered if, when inserting a wire through the lumen portions 34, the wire slides less freely though the lumen portions or, if the structure of the lumen portion is changed more than 50, or more than 10 or more than 2 micrometers.

In FIG. 4, a portion of the fixture 50 used during the welding process is shown. This figure exemplifies a configuration with the bendable body 26 in a horizontal position with the fixture 50 having a plurality of fixture spacers 52 separating the guide rings 36 by a fixed amount defined by the spacing of the fixture spacers 52. The fixture spacers 52 in FIG. 4 are shown as rectangular units, but could alternatively be designed with a round or triangular structure. A laser 54 is positioned to provide light 56 onto the bendable body 26 at the interface between the more transparent guide ring 36 and less transparent inner liner 44. In this exemplary embodiment, the laser 54 is fixed and the bendable body 26 rotates as shown by the arrow 64. As the bendable body 26 rotates, this allows the laser pattern 58 to run around the inside of the ring guide 36/inner liner 44 interface and create a welded portion 60.

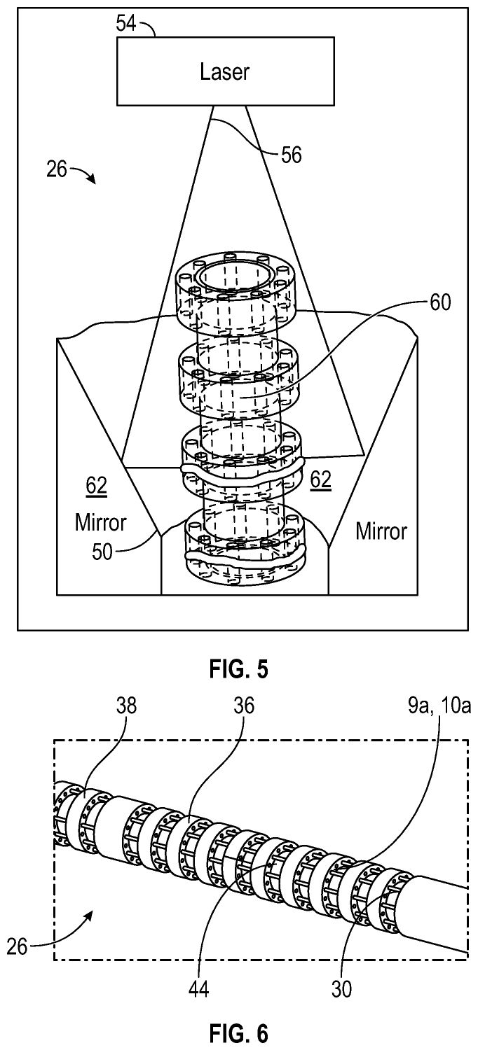

Laser welding often requires a robust physical contact between the materials to be weld. Thus, it is an aspect of the invention to provide tight tolerances to insure such contact. In one exemplary embodiment, a mandrel (not depicted) can be inserted into the hollow chamber 28 (i.e., within the inner liner 44 that is made out of a flexible material, e.g., a material having a flexural modulus between 15 and 55 GPa), such that pressure may be applied from inside the inner liner 44 outward toward the interior of guide rings 36. It is within the scope of this disclosure that the amount of pressure applied from inside the inner liner 44 outward toward the interior of guide rings 36 may be altered based on the size of the mandrel inserted into hollow chamber 28. In some embodiments, a mandrel is paired with an inner liner that can adapt easily to a mandrels geometry and thus insure robust contact between the inner liner and guide rings. In such use, a mandrel will fully expand the diameter of the inner liner creating a tight fit between the inner liner and the guide ring. Additionally, the inner liner may be designed with interference between the inner liner outer diameter and the guide ring inner diameter to insure contact (using the same material properties). The mandrel may remain in the inner liner during the welding process. Further, the mandrel may be rotated to cause the bendable body 26 to rotate, including by automated means to provide repeatable and consistent rotation. An exemplary mandrel should be easily inserted and removed from the hollow chamber 28, and may be made of materials to allow such insertion and removal. In one exemplary embodiment, the mandrel may be coated with a substance having a low coefficient of friction, for instance, Teflon or similar non-stick materials. The welded portion may extend fully or partially around the circumference of the bendable body 26. After welding one guide ring 36 to the inner liner 44, the fixture 50 may be translated as shown by the arrow 62 so that the laser pattern 58 is incident on the bendable body at a second ring guide/inner liner interface. The fixture 50 provided in FIGS. 4 and 5 allow for all parts used to create the bendable body to be held in place and aligned during the welding.

An alternate configuration is to position the bendable body 26 vertically within a concave mirror 62 as shown in FIG. 5. Laser 54 is able to simultaneously weld around a single guide ring 36 with a cylindrical laser pattern 58 without moving the fixture 50 or the bendable body 26. Mirror 62 is also fixed and the bendable body 26 would be pulled through center of mirror 62 to align the various guide rings 36 with the laser pattern 58. In a different embodiment of this configuration, an additional fixture (not shown) is attached to the bendable body to separate the guide rings 36 in a spaced apart relationship prior to performing the laser weld. This fixture can be moved along with the bendable body as the laser weld occurs. In some embodiments, this fixture is substantially transparent to the laser light so as not to absorb the radiation of the weld.

FIG. 6 further depicts a longer portion of bendable body 26, shown with drive rings 38, which may be placed within the sections of guide rings 36, including at the intersection of different bendable sections of bendable body 26. Guide rings 36 and drive rings 38 may be the same or different widths and/or diameters, or may encompass a plurality or range of different widths and/or diameters along a length of the bendable body 26. In one exemplary embodiment, laser welding can be used to weld drive rings 38 to inner liner 44. Also depicted is an extrusion 30, which may be proximal to the one or more different bendable sections of bendable body 26, and through which wires 40a and 42a slidably move through lumen portions of the extrusion 30. Extrusion 30 is bonded to inner liner 44, using laser welding in place of adhesives. Laser welding may be used to first tack components in place to prevent the components from moving out of position before the entire weld is completed.

While FIGS. 1-6 illustrate the distal end of a bendable body (either with or without an outer liner or an extrusion) having the illustrated relative size of inner liner, guide rings, and lumen portions, this method and fixtures can be used for a range of different medical devices with other configurations. It is also contemplated that, more proximal to the portion of the bendable body shown, the bendable body will have a continuous extrusion containing the lumens instead of guide rings. For example, WO 2017/066253, WO 2018/204202, U. S. Pat. Pubs. 2018/0310804, 2018/0243900, 2018/0311006, 2019/0015978, and 2019/0105468 each provide bendable medical instruments, their control, use, and may be made, at least in part, by the methods as provided herein.

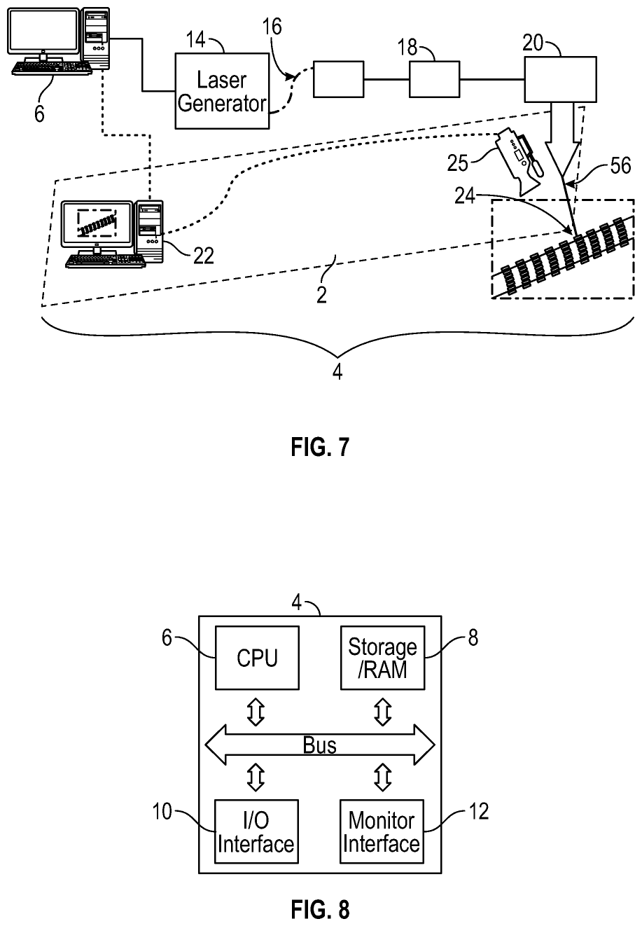

FIG. 7 illustrates an exemplary laser welding system 4, which may include vision system 2. The laser welding system 4 includes a laser generator 14, a transfer fiber 16, a beam shaper 18, a CPU or controller 6, and a galvanometer head 20. The laser generator 14 generates light, or a laser beam 56 by means of a diode laser pump. The transfer fiber 16 transfers the light 56 generated by the laser generator 14 to the beam shaper 28. The beam shaper re-shapes the laser beam and transmits it to the galvanometer head. The galvanometer head moves the laser beam along the weld path. The galvanometer head 29 outputs a laser beam 56 across the welding target 33.

The vision system 2 of the laser welding system 40 may comprise a vision system controller 22 and an optical detector 25. The vision system 2 locates each of the components via the vision system controller 22 which receives input from optical detector 25. The laser welding system 4's controller 6 receives the location of the target component part 24 through a communication port. The controller 6 then moves the axis required of the laser system through PID controls to ensure the laser 56 hits the target component part 24.

It is important to limit any thermal damage to the transparent portion of the device and have melting occur substantially along the inner liner. This can be done by focusing the light at the interface of the inner liner and by defining the relative materials of the guide rings and the inner liner.

Tight control of the power density of the laser 54 is particularly important in some embodiments of the invention. Since energy from the laser will be transmitted through the guide rings and thus the lumen portion, it is important to localize the density at the inner liner interface and provide a weld. In some embodiments, the ring guides are comparatively thicker than the inner liner, which could be a very thin extruded tube. Further, there can be multiple lumen portions located in the ring guides and they can be particularly small and necessarily need to remain homogeneous enough (not deformed) to provide slidability for a wire moving through the lumen portion and providing actuation of the medical device.

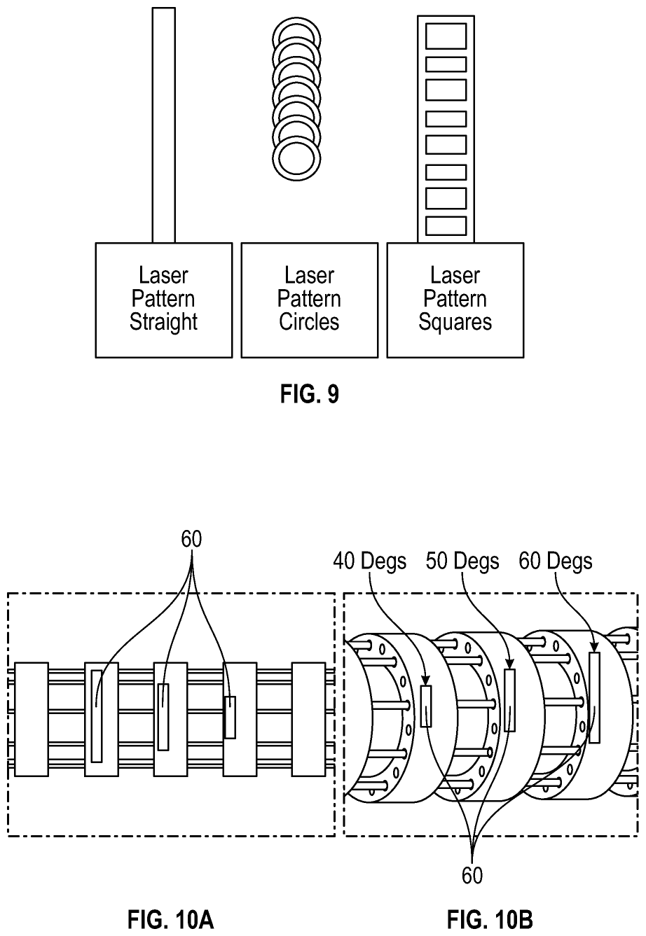

Therefore, in one exemplary embodiment, the laser welding system 4 is configured to utilize those parameters or conditions best suited for the desired weld. Parameters that can be controlled include, but are not limited to: (1) laser power—an exemplary range is from 18 to 30%, and is set by controller 6 and laser generator 14; (2) focal length—an exemplary range is 150 to 200 mm, although this will vary at least part on the target component; the focal length is measured from the of the galvanometer to work piece target component part; (3) clock speed—the speed that the laser beam 54 moves across the work piece, an exemplary range is 20000 to 50000 galvanometer steps/sec; (4) laser passes—the number of times the laser passes over the weld on the target component part; an exemplary range may be 2 to 20 laser passes; (5) clamp pressure—the pressure between mating parts to be welded; the exemplary range can varies based on mandrel diameter, which may be from 0.081″ to 0.088″; (6) welding pattern—the pattern repeated by the laser during lasing, examples of which are seen in FIG. 9, i.e., straight, overlapping circles and overlapping squares; (7) weld time—the total amount of time from weld start to stop, not including positioning/loading of the work piece and/or target component part; ranges are dependent on clock speed and the number of laser passes to be performed; (8) laser wave length—an exemplary range is 1.940 micrometers to 2 micrometers (1940 nanometers to 2000 nanometers); (9) laser type—although other lasers may be suitable, and exemplary laser is a fiber laser; and (10) laser power capability—an exemplary range is up to 120 Watts.

FIG. 4 provides an exemplary embodiment of the manner in which the laser when directed by programming and controls lases the components precisely and repeatedly with the same settings. Use of the fixture 50 allows the weld system 4 to hold the bendable body 26 during lasing, allowing for the tacking of the components in place before the fixture 50 is removed. In one embodiment, a laser weld system 4 can be a 1940 nanometer (2 Micron) Laser System having 5 axis of movement (x-axis (width), laser head rotation, theta-axis (part rotation), y-axis (depth movement) and z-axis (height movement)). The laser system's ability to control motion in the theta-axis (part rotation) can be used in combination with a mandrel as discussed herein such that the motor controls rotation in the theta-axis to precisely rotate the bendable body 26 with an accuracy level to within microns of the desired position.

In combination with the vision system 2, the laser weld system 4 allows for automated movement of the target component and the laser itself, such that the vision system may locate the components and the associated motor controls movement of the laser and/or component in the X, Y, Z, Theta and the Laser Head rotation axis, providing for repeatable accurate and precise welds. Automation of the laser welding, as is provided herein as an exemplary embodiment provides advantages over the use of adhesive in that the welds produced can be designed to have strength characteristics that meet and or exceed performance specifications, while remaining consistent during repetitive iterations on the same or subsequent target components.

The control of the fixture 50, the laser 54, and the bendable body 26 during the weld can be controlled by a computer system as shown in FIG. 7. As more particularly shown in FIG. 8, the computer system 4 includes controller or CPU 6, Storage/RAM 8, I/O Interface 10 and Detector Interface 12. Computer system 4 can be used in communication with vision system 2, or may be used separately. Computer system 4 may comprises one or more devices. For example, the one computer may include components 6, 8, and 10 and other computer may include component 12. The CPU 6 is configured to read and perform computer-executable instructions stored in the Storage/RAM 8. The computer-executable instructions may include those for the performance of the methods and/or calculations described herein. For example, CPU 6 may calculate the amount of rotation and/or translational movement necessary to bring target component part 24 into appropriate alignment with the laser 54 to successfully perform the desired weld.

Storage/RAM 8 optionally includes one or more computer readable and/or writable media, and may include, for example, a magnetic disc (e.g., a hard disk), an optical disc (e.g., a DVD, a Blu-ray), a magneto-optical disk, semiconductor memory (e.g., a non-volatile memory card, flash memory, a solid state drive, SRAM, DRAM), an EPROM, an EEPROM, etc. Storage/RAM 8 may store computer-readable data and/or computer-executable instructions. The components of the computer system 4 communicate via a bus.

The I/O interface 10 provides communication interfaces to input and output devices, which may include a keyboard, a display, a mouse, a printing device, a touch screen, a light pen, an optical storage device, a scanner, a microphone, a camera, a drive, communication cable, sensors such as temperature sensor, and a network (either wired or wireless).

### EXAMPLES

The functional requirements for catheter components are provided in Table 1.

| Description | Requirement |
|---|---|
| Pull strength for | 10-15 Newtons minimum or to material failure |
| extrusions/wire guides | |
| Spacing | on the order of millimeters |
| Orientation | 0 +/− 0.5 degree alignment |
| Alignment (square) | Parallelism of centerlines for wire guides; no |
| | more than 2-3 degrees. |
| End to end twist | Maximum 10 degrees |
| Lumen patency | Lumen remains unaltered |
| Visual test | No bubbles, no over-melting of material, no |
| | holes |
| Leak testing | Hermetically sealed at 25 PSI |

### Example 1: Welding of Inner Cover to Extrusion

The welding of the inner cover to extrusion presented numerous technical challenges: (1) compensating for different laser absorption through areas with or without holes (lumens); (2) the inner cover wall is thin (less than 0.5 mm) and must remain un-breached; (3) extrusion diameter in the range of less than 5 mm; (4) weld must be strong under tension; (4) weld must be hermetically sealed at 25 psi; (5) weld must be done around wires passing through the extrusion.

Welding was performed under variable conditions with and without nitrogen. For tests with nitrogen, N2 was flowed into the inner cover before welding. To perform the welds, the vision system located the extrusion and moved the laser a desired distance past the edge of the extrusion. The extrusion was tacked in place before the parts were rotated to complete the weld around the entire circumference of the parts. 18 separate conditions were tested, in which the number of lines, spacing, number of passes of the laser over the weld seam, weld line width, laser pattern, clock speed (speed the laser beam moves over the weld seam), weld length in degrees and laser power were varied. The laser patterns tested are depicted in FIG. 9. Resulting welds were tested for tensile strength and visually observed. Welds were additionally leak checked.

Overall analysis of the results provided preferred conditions for obtaining welds with the highest tensile strength.

The chosen parameters met both tensile strength requirements and passed leak checks at 25 psi. The use of nitrogen was not selected as it added increased variability to the trials.

### Example 2: Welding of Guide Rings to Inner Liner

The welding of the inner cover to the guide rings presented similar technical challenges as in Example 1: (1) compensating for different laser absorption through areas with or without holes (lumens); (2) the inner cover wall is thin (less than 0.5 mm) and must remain un-breached; (3) guide ring outer diameter is in the range of less than 5 mm; (4) weld must be strong under tension; (4) guide rings are in the range of less than 5 mm wide with corresponding spacing between guide rings; (5) weld must be done around wires passing through the guide rings.

Using the results of Example 1, an experimental design was created to consider 8 conditions: presence of N2, number of spot welds, mumber of passes, spot size, laser pattern, clock speed, weld length (a depiction of how the weld length varies across the guide rings based on 20° between holes (lumens) is shown in FIG. 9) and laser power. Resulting welds were tested for tensile strength and visually observed.

10 additional samples were made and tested for tensile strength at 2 different sites, which tests showed good correlation.

During preparation of the samples, the vision control system enabled the camera to locate and to center the guide rings to center the laser weld pattern. During the centering process, the controls were able to adjust the part in the X and Y axis incrementally in microns, allowing for greater accuracy of positioning. The guide rings were initially tack welded while placed in a holding fixture, after which the fixture was removed to allow the rings to rotate and allow the welding to be performed around the entire circumference of each ring.

Results:

Based on Example 1 and Example 2, preferred settings for laser welding were identified as follows in Table 2

TABLE 2

| Condition | Preferred Setting Ranges |
|---|---|
| Nitrogen (N₂) | Not used |
| Number of Spot Welds | 2-12 |
| Number of Passes | 2-20 |
| Spot Size | .5 mm-2 mm |
| Laser Pattern | Circles, Squares |
| Clock Speed | 20000-50000 |
| Weld Length | 20-80° |
| Laser Power | 18-30 |

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

As used herein the term “catheter” generally refers to a flexible and thin tubular instrument made of medical grade material designed to be inserted through a narrow opening into a bodily lumen (e.g., a vessel) to perform a broad range of medical functions. In some applications a catheter may include a “guide catheter” which functions similarly to a sheath.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. An apparatus comprising:
an inner liner having a hollow chamber extending the length of the inner liner;
at least two guide rings disposed collectively along the inner liner;
at least one lumen portion extending through each of the at least two guide rings and being parallel with the hollow chamber;
wherein the at least two guide rings are welded to the inner liner or wherein the at least two guide rings are fixed by welding.

2. (canceled)

3. The apparatus of claim 1, wherein one lumen portion in each of the at least two guide rings are situated so as to form a lumen in the bendable body such that a wire can be slid through the lumen.

4. (canceled)

5. The apparatus of claim 1, further comprising an outer liner disposed around the at least two guide rings and extending the length of the inner liner.

6. The apparatus of claim 1, comprising at least six guide rings, wherein at least two guide rings each contain at least nine lumen portions.

7. The apparatus of claim 6, further comprising at least nine wires, each extending through at least two guide rings.

8. The apparatus of claim 1 wherein the at least one lumen portion was not noticeably altered by the process of welding the at least two guide rings to the inner liner.

9. The apparatus of claim 1 wherein the weld between the at least two guide rings and the inner liner extends at least half of the circumference of the guide rings inner diameter and inner liner outer diameter.

10. The apparatus of claim 1 wherein the weld between the at least two guide rings and the inner liner extends the full circumference of the guide rings inner diameter and inner liner outer diameter.

11. The apparatus of claim 1 wherein both the inner liner and the at least two guide rings are formed from PEBAX.

12. The apparatus of claim 1, wherein the inner liner contains between 0.1 and 5 percent carbon black.

13. The apparatus of claim 1, comprising a plurality of guide rings disposed collectively along the inner liner.

14. A method of manufacture comprising:
combining at least two guide rings having at least one lumen portion around the outside of an inner liner to create an assembly;
placing the assembly on a fixture adapted to set a distance between each of the at least two guide rings;
welding each of the at least two guide rings to the inner liner;
wherein, the at least two guide rings are substantially transparent and the inner liner is made of a non-transparent light absorbing material.

15. The method of claim 14, wherein the fixture is configured to move linearly at discrete intervals, the intervals being the spacing of the guide rings.

16. The method of claim 14, wherein the fixture is configured to rotate based on a set weld length.

17. The method of claim 14, further comprising inserting a mandrel into the inner liner prior to welding.

18. The method of claim 14, wherein following completion of a weld at a first position on each of the at least two guide rings, the fixture is configured to rotate such that the at least two guide rings are in a second position at which a weld is to be performed.

19. The method of claim 18, wherein the step of performing a weld on each of the at least two guide rings and rotating the at least two guide rings into position for a subsequent weld is repeated until such time as all of the desired welds have been completed.

20. The method of claim 14, wherein the at least two guide rings are made of Pebax.

21. The method of claim 14, wherein the inner liner is made of Pebax with at least 0.5% carbon black.

22. The method of claim 14, wherein welding each of the at least two guide rings to the inner liner requires welding through both lumen containing areas and non-lumen containing areas.

23. A laser welding system comprising:
a vision system;
a laser generator;
a transfer fiber;
a beam shaper;
a galvanometer head;
a motorized fixture; and
a controller in communication with the vision system, the motorized fixture and the laser generator,
wherein, the laser welding system is configured to weld two or more components of a steerable medical device.

24. The system of claim 23, wherein the laser welding system is configured to weld an inner liner having a hollow chamber extending the length of the inner liner and at least two guide rings disposed collectively along the inner liner.

25. The system of claim 23, wherein the laser welding system is configured to weld:
(i) an outer liner to one or more guide rings; or
(2) an extrusion to an inner liner.

26. The system of claim 23, wherein the laser welding system is configured to weld through at least one material having both lumen containing areas and non-lumen containing areas.
